(19) United States
(12) Patent Application Publication
(10) Pub. No.: US 2025/0327792 A1
(43) Pub. Date: Oct. 23, 2025

(54) METHOD FOR PRODUCING MESENCHYMAL STEM CELLS FOR THERAPEUTIC APPLICATIONS

(71) Applicant: REGROW BIOSCIENCES PRIVATE LIMITED, Pune (IN)

(72) Inventors: Satyen SANGHAVI, Pune (IN); Vinayak KEDAGE, Pune (IN)

(21) Appl. No.: 19/184,311

(22) Filed: Apr. 21, 2025

(30) Foreign Application Priority Data
Apr. 22, 2024 (IN) 202421031907

(51) Int. Cl. G01N 33/50; A61K 35/28; C12N 5/0775; G01N 1/30; G01N 15/10

(52) U.S. Cl. CPC G01N 33/5005; A61K 35/28; C12N 5/0663; G01N 1/30; G01N 15/10; G01N 2001/302; G01N 2015/1028

(57) ABSTRACT

The present disclosure relates to a method for producing mesenchymal stem cells for therapeutic applications. The method comprises culturing of MSCs to obtain heterogeneous population. This heterogeneous population of MSCs were subjected to droplet encapsulation media to obtain a population of microfluidic droplets preferably comprising a single MSC; providing the population of microfluidic droplets to a microfluidics device; and identifying and selecting a homogeneous population of MSCs having medium size in the range of 15 to 30 μm showing high expression of cell surface markers (CD73, CD90) and increased expression of genes, such as COL12 A1; IGFBP5; THBS2; GREM1 and CDH2 genes. The MSCs having medium size are further cryopreserved to obtain a cell bank of MSCs with better stability and therapeutic potential in treating diseases like auto immune diseases.

### RELATED APPLICATION

The present application claims priority to Indian Patent Application number 202421031907, filed on Apr. 22, 2024, which is incorporated herein by reference in its entirety.

### FIELD OF INVENTION

The present disclosure relates to the field of mesenchymal stem cells (MSCs) and, particularly to, a method for producing MSCs. It further relates to compositions and therapeutic formulations comprising the MSCs.

### BACKGROUND OF THE INVENTION

Several varieties of drugs are available for treatment of autoimmune diseases like rheumatoid arthritis (RA), which includes steroid hormones, biological agents, immunosuppressant, anti-rheumatic drugs, and anti-inflammatory drugs. However, such approaches have been observed to have undesirable side effects. Reported side effects of RA treatment includes immune deficiency, gastrointestinal tract disorders, hormonal disturbances, and complications in cardiovascular system. Additionally, RA patients in some instances become resistant to long-term treatments. Thus, the current therapeutic approaches for arthritis and similar diseases need an effective alternative involving biological substitutes, which is economical, long effective and having reduced or no side effects.

### SUMMARY OF INVENTION

In an aspect of the present disclosure, there is provided a method of producing mesenchymal stem cells (MSCs) for therapeutic application, the method comprising: a) culturing a heterogeneous population of MSCs in a culture medium for a period in a range of 20 to 35 days; b) staining the MSCs to obtain a heterogeneous population of stained MSCs; c) adding droplet encapsulation media, comprising a density gradient solution to the heterogeneous population of stained MSCs, to obtain a population of microfluidic droplets, wherein each droplet preferably comprises a single MSC; d) providing the population of microfluidic droplets obtained from step (c) to a microfluidics device; and e) identifying and selecting a population of homogeneous MSCs having medium size in the range of 15 to 30 μm; wherein the population of MSCs obtained from step (e) are viable MSCs, expressing MSC specific markers selected from CD 73, CD 90, and combinations thereof; and exhibiting increased expression of genes selected from a group consisting of COL12A gene, IGFBP5 gene, THBS2 gene, GREM1 gene, CDH2 gene, and combinations thereof.

In an aspect of the present disclosure, there is provided a composition comprising the MSCs obtained by the method as disclosed herein; and an excipient; wherein the excipient is selected from DMEM, human serum albumin (HSA), dimethyl sulfoxide (DMSO), fetal bovine serum (FBS), or combinations thereof.

In an aspect of the present disclosure, there is provided a formulation comprising the MSCs obtained by the method as disclosed herein, or the composition as disclosed herein, and a pharmaceutically acceptable carrier.

In an aspect of the present disclosure, there is provided a method of treating a disease in a subject, comprising administering the MSCs obtained by the method as disclosed herein or the formulation as disclosed herein to a subject.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the Wharton's jelly isolation from Umbilical Cord Tissue (UCT): A. UCT (5-7 cm) piece received at manufacturing unit after the complete discharge; B. The cross section of Umbilical Cord tissue, in accordance with the embodiments herein.

FIG. 2 is a schematic representation of the method for producing MSCs, in accordance with the embodiments herein.

FIG. 3 depicts A. Bright field image of single cell encapsulated in a droplet; B. Representative image of an encapsulated cell labelling in deep learning model; and C. Size distribution of MSCs and the Mean fluorescence Intensity measurement of different sized MSCs, in accordance with the embodiments herein.

FIG. 4 depicts the pie chart and bar chart indicating the unique genes present in small and medium sized population, in accordance with the embodiments herein.

FIG. 5 is a representation of gene network of selected 64 genes that were highly expressed in medium sized population when compared to small or large sized population, in accordance with the embodiments herein.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of elements or steps but not the exclusion of any other element or step or group of elements or steps.

The term “including” is used to mean “including but not limited to”. “Including” and “including but not limited to” are used interchangeably.

Embodiments herein include a method for producing MSCs. MSCs are increasingly being used as off the shelf products for treating various diseases. Accordingly, embodiments herein achieve a method for producing MSCs for therapy or therapeutic applications. The method, as disclosed herein, can achieve the production and expansion of medium sized MSCs having therapeutic potential. Further, the embodiments herein also provide a composition comprising the MSCs and an excipient. Embodiments herein also provide a formulation comprising the MSCs and a pharmaceutically acceptable carrier. Accordingly, embodiments herein also include a method for treating a disease in the subject using the composition of MSCs or the formulation.

Embodiments herein provide a method for producing MSCs. The term “mesenchymal stem cell” or “MSCs”, as used herein, refers to a cell or cell population of multipotent cells. The MSCs, according to embodiments herein, are characterized by the expression of one or more cell surface markers selected from CD73, CD90, or CD105. In further embodiments herein, the MSCs may be characterized by not expressing one or more of the markers selected from HLADR, CD34, or CD45.

The MSCs may be obtained from commercial sources or, alternatively, derived from Umbilical Cord Tissue (UCT) of mammals, in particular humans. The MSCs, in accordance with the embodiments herein, are derived from the Wharton's jelly in UCT of single or multiple donors.

In an embodiment of the present disclosure, the heterogeneous population of MSCs is obtained from human umbilical cord tissue (UCT) of single donor or multiple donors.

In some embodiments, the donor for obtaining UCT are selected based on a set of physiological and medical history parameters. In an embodiment, the UCT donor is a healthy pregnant female with a negative association with the medical history parameters selected from drug addiction; alcohol addiction; chronic heart and respiratory diseases; malignancies; organ, tissue, or any past stem cell transplant; past history of diabetes; joint arthroplasty; viral/vector borne diseases, such as dengue, CJD, etc; hospitalization or surgery in the past year; cupping or tattooing in the past year; dentistry in the past week; bypass/angiography or haemodialysis; high-risk sexual behaviors; domestic or international travel history to a high-risk area in terms of infectious disease; bleeding problems; blood pressure abnormalities; sepsis and fever of unknown origin; liver disease; autoimmune disease; hepatitis with unknown cause; genital ulcerative disease; disseminated lymphadenopathy and splenomegaly; bruising or skin lesions; neurological disorders; metabolic disorders or genetic disorders. In further embodiments, the selected donor is vaccinated as per national vaccination schedule.

In some embodiments, the UCT donor is a healthy pregnant female with an acceptable criterion for physiological parameters identified through blood, serum, and urine analysis. The various physiological parameters and the corresponding acceptance criterion for selecting a donor for MSCs isolation is listed in Table A below.

TABLE A
Blood, serological and urine analyses

| | Test Parameter | Accepted criteria | |
|---|---|---|---|
| 1 | HLA typing | Must be done | |
| 2 | Fasting plasma glucose test | 60-100 | mg/dl |
| | (FPG) | | |
| 3 | CRP | Negative | |
| 4 | Hb | No Hb variant detected | |
| 5 | Electrolytes | 135-145 | mEq/L |
| | Na | 3.5-5.3 | mEq/L |
| | K | 8.3-1.3 | mg/dl |
| | Ca | | |
| 6 | Urea | 11-50 | mg/dl |
| 7 | Creatinine | 0.5-1.4 | mg/dl |
| 8 | Albumin | 3.5-5.2 | g/dl |
| 9 | Total bilirubin (direct and | 0.3-1.2 | mg/dl |
| | indirect) | | |
| 10 | Urinalysis | Normal | |
| 11 | Alkaline phosphatase | 64-306 | U/L |
| 12 | PT | 12-14.5 | seconds |
| 13 | PTT | 22-37 | seconds |
| 14 | SGOT | <45 | U/L |
| 15 | SGPT | <45 | U/L |
| 16 | LDH | 100-190 | U/L |
| 17 | CBC with differential | Normal | |
| 18 | HIV ½ | Negative | |
| 19 | HTLV ½ | | |
| 20 | HBV | | |
| 21 | HCV | | |
| 22 | CMV | | |
| 23 | EBV | | |
| 24 | RPR | Negative | |
| 25 | Serum beta-HCG pregnancy | <5 | mIU/Ml |

Pregnancy relevant test

| | | |
|---|---|---|
| 1 | Multiple marker test | Negative |
| 2 | Ultrasound scan | Normal |
| 3 | Alpha fetoprotein screening | Normal |
| 4 | Delivery | Full term (37-42 weeks) |
| 5 | Surrogacy case | NA |

In certain embodiments, the MSCs are isolated from UCT. Various methods are known in the art for the isolation of MSCs from UCT.

In an embodiment, the UCT are minced and subjected to enzymatic digestion. In an embodiment, the heterogeneous population of MSCs is obtained by treating the human umbilical cord tissue with at least one enzyme selected from collagenase, hyaluronidase, proteins identified from CB+MB (Cord Blood and Maternal Blood) plasma lysate [Peroxiredoxin 1 (PRDX1) and Heat Shock Protein-70 (HSP70)], or mixtures thereof. In another embodiment, the heterogeneous population of MSCs is obtained by treating the human umbilical cord tissue with collagenase, hyaluronidase, and proteins identified from CB+MB plasma lysate (PRDX1 and HSP70). Collagenase and hyaluronidase may be procured commercially from Thermofisher, Catalogue Number: 17100017 and Sigma-Aldrich, Catalogue Number: H3506, respectively. Various methods well known in the art can be used to obtain the proteins, such as Peroxiredoxin 1 (PRDX1) and Heat Shock Protein-70 (HSP70 from cord blood and maternal blood plasma. For example, the plasma proteins may be precipitated from the mixture of cord blood and maternal blood obtained from the same donor using protein precipitating agents, such as ammonium acetate, ethanol, or combination thereof.

The MSCs isolated from UCT may be a heterogeneous population of MSCs. The term “heterogeneous population”, as used herein, refers to a mixed population of small, medium, or large sized MSCs. According to embodiments herein, the MSCs having medium size are particularly expanded and produced using the method as described herein. The term “expanded” or “expanded population”, as used herein, refers to an increased population of cells, wherein the number of cells is higher as compared to before expansion. It refers to a multiplied cell population.

In an embodiment, there is provided a method of producing mesenchymal stem cells (MSCs) for therapeutic application, the method comprising: a) culturing a heterogeneous population of MSCs in a culture medium for a period in a range of 20 to 35 days; b) staining the MSCs to obtain a heterogeneous population of stained MSCs; c) adding droplet encapsulation media, comprising a density gradient solution to the heterogeneous population of stained MSCs, to obtain a population of microfluidic droplets, wherein each droplet preferably comprises a single MSC; d) providing the population of microfluidic droplets obtained from step (c) to a microfluidics device; and e) identifying and selecting a homogeneous population of MSCs of medium size in the range of 15 to 30 μm; wherein the population of MSCs obtained from step (e) are viable MSCs, expressing MSC specific markers selected from CD 73, CD 90, and combinations thereof; and exhibiting increased expression of genes selected from a group consisting of COL12A gene, IGFBP5 gene, THBS2 gene, GREM1 gene, CDH2 gene, and combinations thereof.

Culturing, according to embodiments herein, refers to growing or maintaining MSCs by providing the physical conditions (for eg: temperature) and chemical conditions (for eg: buffers, growth factors, nutrients, vitamins, etc).

The medium for culturing MSCs, according to the present disclosure, is a medium capable of proliferating MSCs. Various MSCs culture media are known and may be used in embodiments herein. Examples of MSC culture media include, but are not limited to, Dulbecco's Modified Eagle Medium (DMEM), Iscove's Modified Dulbecco's Medium (IMDM), DMEM-F12, F12, Minimum Essential Medium a (ALPHA-MEM), etc. In an embodiment, the culture medium is selected from DMEM, IMDM, DMEM-F12, F12, ALPHA-MEM, or combinations thereof. In another embodiment, the medium is Dulbecco's Modified Eagle Medium (DMEM) (Thermo Fisher) with 10% of Fetal Bovine Serum (FBS) (EDQM Certified), BMP4 and Ing to 10 ng per ml of Fibroblast Growth Factor (FGF) (Thermofisher).

The MSCs culture medium may be replaced every 2-7 days. In an embodiment, the MSCs culture medium is replaced every 2 to 7 days. In another embodiment, the MSCs culture medium is replaced every 2 to 3 days, 2 to 4 days, 2 to 5 days, 2 to 6 days, or 2 to 7 days.

In some embodiments, the heterogeneous MSC population is cultured to a confluence of at least 80%, but less than 100%, more preferably the heterogeneous MSC population is cultured to a confluence of 80% to 90%.

Upon achieving the desired confluency, the MSCs may be trypsinized to detach the suspended cells from the culture flasks and reseeded for adherence. The culturing and optional trypsinization can be repeated so that the MSC-containing population of cells achieves a desired passage number. Typically, the initial population of cells P0 (passage 0) is the cells that have been derived from UCT. When P0 is defined as the extracted population, then the MSCs expanded up to P3 or P4 is sufficient to obtain enough medium-sized MSCs.

According to embodiments herein, passage 3 (P3) is adequate to produce a MSC population with therapeutic potential.

In an embodiment of the present disclosure, the step (a) further comprises reseeding the heterogeneous population of MSCs at a cell density in the range of 5000 to 11,000/cm² and expanding the heterogeneous population of MSCs up to passage 3.

After every passage, the MSCs may be monitored for its attachment status, cell morphology, cell growth and microbial contamination, if any. The remaining MSCs may be cryopreserved using suitable cryostoring solutions, for eg: Cryostor™ containing 10% DMSO. In an embodiment, cryopreservation is performed in cryotubes containing 10% DMSO, at a temperature in the range −75 to −85° C., preferably −80° C. temperature or in liquid nitrogen tanks. The cryopreserved MSCs can be thawed later for further expansion.

In an embodiment, the heterogeneous population of MSCs in step (b) are stained using a cell viability stain or MSC marker stain.

Various methods are known in the art to assess the viability status of culture expanded MSCs. For example, MSCs are stained using certain cell viability stains that help in differentiating the viable MSCs and non-viable MSCs. Similarly, various MSC marker stains are known in the art to determine the characteristic expression of MSC cell surface markers, and to confirm the identity of MSCs. In an embodiment, the cell viability stain is selected from carboxyfluorescein diacetate succinimidyl ester (CFSE), trypan blue, propidium iodide (PI), or combinations thereof; and the MSC marker stain is selected from CD73, CD90, CD105, or combinations thereof; and wherein the heterogeneous population of MSCs is having a concentration in the range of 0.5 to 5×10⁶ cells/ml, preferably having a concentration of 1×10⁶ cells/ml.

The MSCs of P3 are validated for their viability and MSC marker status and may be further subjected to sorting or for particularly separating the medium sized MSCs from the culture expanded heterogeneous population of MSCs. MSCs can be fractioned to small, medium, or large-sized MSC populations by any modality suitable to isolate these populations from an MSC-containing population, such as a heterogeneous MSC population. Exemplary modalities for separation of medium-sized MSCs include microfluidic devices, size exclusion filters, and density columns. According to embodiments herein, the heterogeneous MSCs are processed in a microfluidics platform to separate out the medium-sized MSCs. Examples of microfluidics platform that are well known in the art, that could be used are droplet, capillary, pressure, acoustics, or electrical based microfluidics platforms. In an embodiment, the microfluidics device is droplet microfluidic platform.

In an embodiment, the heterogeneous population of stained MSCs are encapsulated in a droplet, by a droplet generation device. According to embodiments herein, a droplet encapsulation media is added to the heterogeneous population of stained MSCs to obtain a population of microfluidic droplets, wherein each droplet preferably comprises a single MSC.

In an embodiment, the droplet encapsulation media comprises a density gradient solution, wherein the density gradient solution is selected from an iodixanol density gradient solution or a sucrose gradient solution. In another embodiment, the droplet encapsulation media comprises a density gradient solution, wherein the density gradient solution is an iodixanol density gradient solution (Opti-prep density gradient solution). The droplet encapsulation media further comprises a MSCs culture medium, such as DMEM as described hereinabove to maintain the viability of MSCs during droplet generation. Examples of droplet generation device that are known in the art that could be used are active droplet formation (electric, magnetic, centrifugal) and passive droplet formation (cross-flowing, flow focusing, co-flowing) devices.

In an embodiment, the size of the microfluidic droplet is in the range of 100 to 200 micron; and each of the microfluidic droplet is sufficient to allow the MSCs to replicate 3 to 4 times. In another embodiment, the size of the microfluidic droplet is in the range of 120 to 150 micron.

In an embodiment, the step of the disclosed process further comprises imaging the MSCs in the population of microfluidic droplets to record the size of the MSCs; and subjecting the population of microfluidic droplets to automated size-based sorting to obtain a population of homogeneous MSCs having medium size.

The microfluidic droplets are sorted or analysed in a microfluidic device and imaged using microscopy. Various microscopy techniques that can be used to image the droplets are fluorescence microscopy, confocal microscopy, optical microscopy, electron microscopy etc., In an embodiment, the microscopy technique used for imaging MSCs is bright field fluorescence microscopy.

In an embodiment, the microfluidic droplets are analysed at a flow rate in the range of 100 to 200 nanolitre/sec, preferably at a flow rate of 150 nanolitre/sec.

In certain embodiments, the stained heterogeneous population of MSCs are sorted based on the cell size of the encapsulated MSCs, their cell viability and MSC marker stain status. The sorted droplets are lodged into nanowells. In an embodiment, 100 microfluidic droplets are lodged into nanowells of the microfluidic device.

In an embodiment, the population of microfluidic droplets are subjected to automated size-based sorting to obtain a homogeneous population of MSCs having medium size. The automated size-based sorting may be performed using various methods known in the art, such as using artificial intelligence (AI)-based tool.

In an embodiment, the medium-sized MSCs are sorted from the population of microfluidic droplets using automated size-based sorting, preferably using an artificial intelligence-based tool trained for size-based identification of cells.

According to the present disclosure, the artificial intelligence-based tool for automated size-based sorting of MSCs is developed using python algorithms to train the model in size-based sorting of small, medium- and large sized MSCs. Alternatively, Fluorescence Activated Cell Sorting using flow cytometry could also be used for size-based sorting of MSCs.

The term “medium-sized MSCs” as used herein refers to a population of MSCs characterized by the size in the range of 15 to 30 μm. The term “small-sized MSCs” as used herein refers to a population of MSCs characterized by having a size less than 15 μm, preferably 12.2±1.9 μm. The term “large-sized MSCs” as used herein refers to a population of MSCs characterized by having a size greater than 30 μm, preferably, 35.8±6.7 μm.

In an embodiment, the population of MSCs of medium size is characterized by having a size in the range of 15 to 30 μm. In another embodiment, the population of MSCs of medium size is characterized by having a size in the range of 17 to 22 μm.

In an embodiment, the homogeneous population of MSCs comprises at least 50%, at least 58% to 60%, at least 70%, or at least 80% of medium-sized MSCs.

In further embodiments, the medium-sized MSCs are characterized by the expression of certain genes that aid their therapeutic potential. In an embodiment, the medium-sized MSCs are characterized by the increased expression of genes selected from COL12A, IGFBP5, THBS2, GREM1, CDH2, CLDN11, POSTN, COL11A1, CDH11, CCL2, SLC7A2, COL8A1, GJA1, CCDC80, ACTG2, ANTXR1, THSD4, RND3, MXRA5, NTM, TEAD1, YAP1, CSPG4, RP11-166D19.1, TPBG, FAP, LAMC2, LUM, NUAK1, RAB3B, GPX8, DSC3, LRRC17, GPC6, FBLN1, ADAMTS1, NR2F2, SEMA5A., SLIT2, TBX3, LARP6, HAS2, FAM101A, FLRT2, CD276, NUPR1, TJP1, PTPN13, EVC, KCNG1, PAMR1, PDLIM3, STC1, SCARA3, PRRX1, GPC1, SSTR1, DDX3Y, MOXD1, RGS4, TBX2, CPA4, IGFBP6, VAT1L, or combination thereof. In another embodiment, medium-sized MSCs are characterized by the increased expression of genes selected from a group consisting of COL12A gene, IGFBP5 gene, THBS2 gene, GREM1 gene, CDH2 gene, and combinations thereof.

In an embodiment, the method further comprises, expanding the medium size MSCs up to passage 6 or up to passage 20 in culture; and optionally cryopreserving the medium size MSCs. The cryopreserved MSCs are also referred to herein as “cell bank”. The cell bank of medium-sized MSCs can be thawed to be used for therapy or in therapeutic applications as such or in the form of a composition or formulation according to various embodiments herein.

Embodiments herein provide a composition comprising the medium-sized MSCs obtained by the method described in the present disclosure.

In an embodiment, there is provided a composition comprising the MSCs obtained by the method as disclosed herein; and an excipient; wherein the excipient is selected from DMEM, human serum albumin (HSA), dimethyl sulfoxide (DMSO), fetal bovine serum (FBS), cord blood and maternal blood plasma or combinations thereof.

Embodiments herein provide a formulation comprising the medium-sized MSCs obtained by the method described in the present disclosure, or the composition as disclosed herein.

In an embodiment, there is provided a formulation comprising the MSCs obtained from the method as described herein, or the composition as disclosed herein, and a pharmaceutically acceptable carrier.

The term “pharmaceutically acceptable carrier” as used herein refers to any carrier including adjuvants, solvents, etc. that are suitable for preparing therapeutic stem cell formulations. Various carriers are known to a person skilled in the art and may be used in various embodiments herein. In an embodiment, the pharmaceutically acceptable carrier is selected from ringer lactate solution, saline, dextrose, heparin, or combinations thereof.

Embodiments herein further include a method of treating a disease in a subject.

The term “subject”, as used herein, refers to mammals, e.g., human and non-human mammals. Examples of non-human animals include non-human primates, dogs, cats, cattle, horses, sheep, pigs, goats, rabbits, mice, rats, hamsters, guinea pigs and etc. Unless otherwise noted, the terms “patient” or “subject” are used herein interchangeably. Preferably, the subject is human.

In an embodiment, there is provided a method of treating a disease in a subject, comprising administering the MSCs obtained from the method as described herein or the formulation as disclosed herein to a subject.

In an embodiment, the MSCs are allogenic to the subject.

In an embodiment, the disease is selected from a group consisting of autoimmune diseases, autoimmune diseases (Rheumatoid Arthritis (RA), Acquired Aplastic Anemia, Acquired Hemophilia, Agammaglobulinemia, Alopecia Areata, Ankylosing Spondylitis (AS), Anti-NMDA Receptor Encephalitis, Antiphospholipid Syndrome (APS), Arteriosclerosis, Autoimmune Addison's Disease (AAD), Autoimmune Autonomic Ganglionopathy (AAG), Autoimmune Encephalitis (AE)/Acute Disseminated Encephalomyelitis (ADEM), Autoimmune Gastritis, Autoimmune Hemolytic Anemia (AIHA), Autoimmune Hepatitis, Autoimmune Hyperlipidemia, Autoimmune Hypophysitis/Lymphocytic Hypophysitis, Autoimmune Inner Ear Disease (AIED), Autoimmune Lymphoproliferative Syndrome (ALPS), Autoimmune Myelofibrosis (AIMF), Autoimmune Myocarditis, Autoimmune Oophoritis, Autoimmune Pancreatitis (AIP), Autoimmune Polyglandular Syndromes (APS), Autoimmune Progesterone Dermatitis (APD), Autoimmune Retinopathy (AIR), Autoimmune Sudden Sensorineural Hearing Loss, Balo Disease/Concentric Sclerosis, Behçet's Disease, Birdshot Chorioretinopathy/Birdshot Uveitis, Bullous Pemphigoid, Castleman Disease, Celiac Disease, Chagas Disease, Chronic Inflammatory Demyelinating Polyneuropathy (CIDP), Chronic Autoimmune Urticaria, Churg-Strauss Syndrome/Eosinophilic Granulomatosis with Polyangiitis (EGPA), Cogan's Syndrome (CS), Cold Agglutinin Disease (CAD), Crest Syndrome, Crohn's Disease, Stricturing Crohn's Disease, Cronkhite-Canada Syndrome (CCS), Cryptogenic Organizing Pneumonia (COP), Dermatitis Herpetiformis (DH), Dermatomyositis, Diabetes, Type 1 (TID), Discoid Lupus Erythematosus (DLE), Dressler's Syndrome/Post myocardial Infarction/Post pericardiotomy Syndrome, Eczema/Atopic Dermatitis, Eosinophilic Fasciitis, Erythema Nodosum, Essential Mixed Cryoglobulinemia, Evans Syndrome, Fibrosing Alveolitis/Idiopathic Pulmonary Fibrosis (IPF), Giant Cell Arteritis/Temporal Arteritis/Horton's Disease, Giant Cell Myocarditis, Glomerulonephritis (GN), Goodpasture's Syndrome/Anti-Gbm/Anti-Tbm Disease, Granulomatosis With Polyangiitis (GPA)/Wegener's Granulomatosis, Graves' Disease (GD), Guillain-Barre Syndrome (GBS), Hashimoto's Thyroiditis/Autoimmune Thyroiditis, Henoch-Schölein Purpura (HSP)/Iga Vasculitis, Hidradenitis Suppurativa, Hurst's Disease/Acute Hemorrhagic Leukoencephalitis (AHLE), Hypogammaglobulinemia, Iga Nephropathy/Berger's Disease, Immune-Mediated Necrotizing Myopathy (IMNM), Immune Thrombocytopenia (Itp)/Autoimmune Thrombocytopenia Purpura, Inclusion Body Myositis (IBM), Igg4-Related Sclerosing Disease (ISD), Interstitial Cystitis, Juvenile Idiopathic Arthritis (Jia)/Adult-Onset Still's Disease, Juvenile polymyositis/Juvenile dermatomyositis/juvenile myositis, Kawasaki disease, Lambert-Eaton Myasthenic Syndrome (LEMS), Leukocytoclastic vasculitis, Lichen Planus, Lichen Sclerosus, Ligneous conjunctivitis, Linear Iga Disease (LAD), Lupus Nephritis (LN), Lyme Disease/Chronic Lyme Disease/Post-Treatment Lyme Disease Syndrome (PTLDS), Lymphocytic colitis/microscopic colitis, Lymphocytic hypophystitis/autoimmune hypophystitis, Ménière's Disease, Microscopic Polyangiitis (MPA)/ANCA-Associated Vasculitis, Mixed Connective Tissue Disease (MCTD), Mooren's ulcer, Mucha-Habermann disease, Multifocal motor neuropathy, Multiple Sclerosis (MS), Myalgic Encephalomyelitis/Chronic Fatigue Syndrome (ME/CFS), Myasthenia Gravis (MG), Narcolepsy, Neuromyelitis Optica/Devic's Disease, Ocular Cicatricial Pemphigoid, Opsoclonus-myoclonus syndrome (OMS), Palindromic Rheumatism, Paraneoplastic Cerebellar Degeneration (PCD), Paraneoplastic Pemphigus, Parry-Romberg Syndromeherth (PRS)/Hemifacial Atrophy (HFA)/Progressive Facial Hemiatrophy, Paroxysmal Nocturnal Hemoglobinuria (PNH), Peripheral uveitis/pars planitis, PANS/PANDAS, Parsonage-Turner Syndrome (PTS), Pemphigoid Gestationis (PG), Pemphigus Foliaceus, Pemphigus Vulgaris, Pernicious anemia, POEMS Syndrome, Polyarteritis Nodosa (PAN), Polymyalgia Rheumatica, Polymyositis, Postural Orthostatic Tachycardia Syndrome (Pots), Primary Biliary Cirrhosis (PBC), Primary Sclerosing Cholangitis (PSC), Psoriasis, Palmoplantar Pustulosis (PPP), Psoriatic Arthritis, Pulmonary fibrosis, idiopathic (IPF), Pure Red Cell Aplasia (PRCA), Pyoderma gangrenosum, Rasmussen's encephalitis, Raynaud's Syndrome, Reactive Arthritis, Reflex sympathetic dystrophy syndrome (RSD)/Complex regional pain syndrome (CRPS), Relapsing Polychondritis (RP), Restless leg syndrome (RLS)/Willis-Ekbom disease, Rheumatic Fever, Sarcoidosis, Schmidt Syndrome/Autoimmune Polyendocrine Syndrome Type II, Scleritis, Scleroderma, Sclerosing Mesenteritis/Mesenteric Panniculitis, Serpiginous choroidopathy, Sjögren's Syndrome, Stiff person syndrome (SPS), Small Fiber Sensory Neuropathy (SFSN), Small Fiber Sensory Neuropathy (SFSN), Systemic Lupus Erythematosus (SLE), Subacute bacterial endocarditis (SBE), Subacute cutaneous lupus, Susac's syndrome, Sydenham's Chorea, Sympathetic ophthalmia, Takayasu's arteritis (vasculitis), Testicular Autoimmunity, Tolosa-Hunt syndrome, Transverse myelitis™, Tubulointerstitial nephritis uveitis syndrome (TINU), Ulcerative Colitis, Undifferentiated Connective Tissue Disease, Uveitis, Vasculitis, VEXAS Syndrome, Vogt-Koyanagi-Harada syndrome (VKH), Osteoarthritis, AVN, vertebral compression factor, urethral stricture, Sjogren's syndrome and ureteric stricture), arthritis, Type I Diabetes, multiple sclerosis, inflammatory bowel diseases and acromegaly.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### Examples

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may vary.

### Example 1: Isolation of MSCs from Umbilical Cord Tissue (UCT)

### 1. UCT Donor Selection and Evaluation

The umbilical cord tissue (UCT) sample was collected from a healthy volunteer (pregnant females) after receipt of written informed consent, which was obtained according to ISSCR (ICMR) guidelines and approval of the Institutional Ethics Committee, Regrow Biosciences Pvt. Ltd. for the use of intended human biological sample for manufacturing and therapeutic goals (Reg. No: ECR/309/Inst/MH/2019/RR-22).

Eligible, healthy volunteers for UCT were carefully chosen according to the inclusion criteria and exclusion criteria approved by Institutional Ethics Committee, Regrow Biosciences Pvt. Ltd (Table 1). Briefly donor competency referred to general health through asking volunteer's medical history, physical examination, serological testing to insure the medical health and absence of viral infections, chest X-ray and electrocardiogram. The health certificate from the physician was necessary to confirm the qualification of the eligible donor.

TABLE 1
Medical eligibility and suitability of UCT females
Donor medical history

| | History and physical | |
|---|---|---|
| Sr. No. | examination | Accepted criteria |
| 1 | Drug addiction | Negative |
| 2 | Alcohol addiction | Negative |
| 3 | Chronic heart and respiratory | Negative |
| | diseases | |
| 4 | Malignancies | Negative |
| 5 | Organ, tissue, or any post stem | Negative |
| | cell transplant | |
| 6 | Vaccination | According to national |
| | | vaccination schedule |
| 7 | General anaesthesia | Negative |
| 8 | Hospitalization or surgery in the | Negative |
| | past year | |
| 9 | Cupping or tattooing in the past | Negative |
| | year | |
| 10 | Dentistry in the past week | Negative |
| 11 | Bypass/Angiography or | Negative |
| | haemodialysis | |
| 12 | High-risk sexual behaviors | Negative |
| 13 | Domestic or international travel | Negative |
| | history to a high-risk area in | |
| | terms of infectious disease | |
| 14 | Bleeding problems | Negative |
| 15 | Blood pressure | Negative |
| 16 | Sepsis and fever of unknown | Negative |
| | origin | |
| 17 | Liver disease | Negative |
| 18 | Autoimmune diseases | Negative |
| 19 | Hepatitis with unknown cause | Negative |
| 20 | Genital ulcerative disease | Negative |
| 21 | Disseminated lymphadenopathy | Negative |
| | and splenomegaly | |
| 22 | Bruising or skin lesions | Negative |
| 23 | Neurological disorders/conditions | Negative |
| 24 | Metabolic disorders | Negative |
| 25 | Genetic disorders | Negative |

Blood, serological and urine analyses

| | Test | Accepted criteria | |
|---|---|---|---|
| 1 | HLA typing | Must be done | |
| 2 | Fasting plasma glucose test | 60-100 | mg/dl |
| | (FPG) | | |
| 3 | CRP | Negative | |
| 4 | Hb | No Hb variant detected | |
| 5 | Electrolytes | 135-145 | mEq/L |
| | Na | 3.5-5.3 | mEq/L |
| | K | 8.3-1.3 | mg/dl |
| | Ca | | |
| 6 | Urea | 11-50 | mg/dl |
| 7 | Creatinine | 0.5-1.4 | mg/dl |
| 8 | Albumin | 3.5-5.2 | g/dl |
| 9 | Total bilirubin (direct and | 0.3-1.2 | mg/dl |
| | indirect) | | |
| 10 | Urinalysis | Normal | |
| 11 | Alkaline phosphatase | 64-306 | U/L |
| 12 | PT | 12-14.5 | seconds |
| 13 | PTT | 22-37 | seconds |
| 14 | SGOT | <45 | U/L |
| 15 | SGPT | <45 | U/L |
| 16 | LDH | 100-190 | U/L |
| 17 | CBC with differential | Normal | |
| 18 | HIV ½ | Negative | |
| 19 | HTLV ½ | | |
| 20 | HBV | | |
| 21 | HCV | | |
| 22 | CMV | | |
| 23 | EBV | | |
| 24 | RPR | Negative | |
| 25 | Serum beta-HCG pregnancy | <5 | mIU/mL |

Pregnancy relevant test

| | | |
|---|---|---|
| 1 | Multiple marker test | Negative |
| 2 | Ultrasound scan | Normal |
| 3 | Alpha fetoprotein screening | Normal |
| 4 | Delivery | Full term (37-42 weeks) |
| 5 | Surrogacy case | NA |

### 2. Umbilical Cord Tissue Collection, Transportation, and Suitability Testing

Umbilical cord tissue samples under a sterile condition were taken from full-term and healthy female new-borns by normal/caesarean section. The umbilical cord tissue was transferred to the cell manufacturing unit, within 72 h in phosphate buffered saline (PBS, HiMedia) at 15-28° C. Approximately, 10-15 cm length and 6 to 9 mm thick umbilical cord tissue was cut into two and transferred into a pre-labelled sterile container containing 20 ml of (1X) DPBS buffer solution supplemented with penicillin (100 U/ml), streptomycin (100 μg/ml) and gentamycin (Gibco) (10 μg/ml). Maternal and Cord blood (2 ml each) were also collected in EDTA vial for reconfirming the Transfusion transmissible diseases such as infectious diseases such as HIV 1 & 2 antibodies, HCV antibodies, Anti-HBc, HBsAg, HTLV 1 & 2, Syphilis antibodies, Malarial parasite, CMV IgM and IgG (Table 2).

TABLE 2
Transfusion transmissible disease screening and karyotyping
Transfusion transmissible diseases tests

| Test | CT-MSC-01 | CT-MSC-02 | CT-MSC-03 |
|---|---|---|---|
| HIV 1&2 | Negative | Negative | Negative |
| antibodies | | | |
| HCV antibodies | Non Reactive | Non Reactive | Non Reactive |
| Anti-HBC | Non Reactive | Non Reactive | Non Reactive |
| HBsAg | Non Reactive | Non Reactive | Non Reactive |
| HTLV 1&2 | Negative | Negative | Negative |
| Syphilis | Non-Reactive | Non-Reactive | Non-Reactive |
| antibodies | | | |
| Malarial parasite | Not Detected | Not Detected | Not Detected |
| CMV IgM/IgG | Negative/Immune | Negative/Immune | Negative/Immune |

Other tests

| | | | |
|---|---|---|---|
| Karyotyping | Normal | Normal | Normal |

3. Isolation of MSCs from Umbilical Cord Tissue

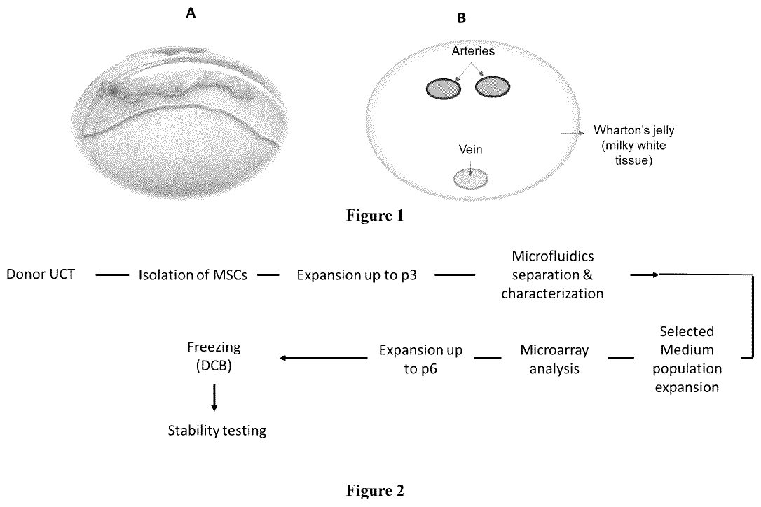

After obtaining Umbilical Cord tissue, the blood cells, arteries, and vein were removed from the umbilical cord tissue (FIGS. 1A & B) and the remaining Wharton's jelly were minced into small pieces. Briefly, the Umbilical Cord tissue was cut into 1-2 cm sized pieces after removal of the blood vessel and repeated washing was done with (1X) Ddpbs

buffer solution supplemented with antibiotics 100 U/ml of penicillin (Invitrogen), 100 μg/ml of streptomycin (Invitrogen) and 10 μg/ml of gentamicin (Gibco) in a biosafety cabinet. Half of these pieces were incubated with 1-5 mg/ml Collagenase type-1 (Gibco) and 2-3 mg/ml hyaluronidase (Sigma) and rest half were incubated with 1-5 mg/ml Collagenase type-1 (Gibco) and 2-3 mg/ml hyaluronidase (Sigma) along with proteins identified from CB+MB plasma lysate (PRDX and HSP70) for 2-6 hours at 37° C. on shaking incubator. After complete digestion of tissue, the samples containing enzyme solution (treated umbilical cord matrix reaction solution) were filtered by using a 40-100 μm cell strainer, to obtain a filtrate solution. The filtrate solution (filtered solution) was centrifuged at 1100-1800 rpm for 5-10 minutes. The supernatant were discarded, and the pellet were dissolved in Dulbecco's Modified Eagle Medium (DMEM) (Thermo Fisher) with 10% of Fetal Bovine Serum (FBS) (EDQM Certified), BMP4 and 1 ng to 10 ng per ml of Fibroblast Growth Factor (FGF) (Thermofisher), preferably 5 ng/ml to obtain a stem cell suspension. The stem cell suspension was seeded into cell culture flask followed by incubation at 37° C. in a humidified atmosphere with 5% CO₂ to obtain a culture flask-adhered cultured stem cells. The culture flask-adhered cultured stem cells were allowed to expand until it reached about 70-80% confluence. Morphology of isolated and expanded mesenchymal stem cells (MSCs) was assessed using an inverted microscope, and the results were recorded for cell count, cell viability and colony forming unit (CFU) (Table 3). It is evident that the use of a combination of enzymes, such as Collagenase and Hyaluronidase along with proteins identified from CB+MB plasma lysate (PRDX1 and HSP70) in the enzymatic digestion of UCT resulted in improved viability of the isolated MSCs.

### 4. Procedure for Obtaining CFUs:

About 100 MSCs were plated per 100-mm tissue culture dish (Falcon) in a complete culture medium. Then the cells were incubated for 10-14 days at 37° C. in 5% humidified CO2. The media was removed after 14 days, washed with PBS, and stained with 0.5% crystal violet in methanol for 5-10 min at room temperature. Following which, the plates were washed with PBS twice and the MSCs colonies that were violet colored were observed under the microscope and the colonies were counted.

TABLE 3
Enzymatic isolation procedure and assessment of cell count, viability and
CFU F (Colony Forming Unit-Fibroblast).

| | | Test Parameter | | | | | | | | |
|---|---|---|---|---|---|---|---|---|---|---|
| | | | | | | | | MSC CFU | | |
| | | Cell Count (In10\hat{ }6) | | | Cell Viability (In %) | | | (No of colonies) | | |
| | | Sample ID | | | Sample ID | | | Sample ID | | |
| | | CT- | | CT- | CT- | | CT- | CT- | | CT- |
| | Test | MSC- | CT-MSC- | MSC- | MSC- | CT-MSC- | MSC- | MSC- | CT-MSC- | MSC- |
| S. No | Combination | 01 | 02 | 03 | 01 | 02 | 03 | 01 | 02 | 03 |
| 1 | Enzymatic | 4.2 | 4.0 | 4.3 | 94.5 | 95.1 | 94.9 | 3 | 5 | 4 |
| | digestion | | | | | | | | | |
| | using | | | | | | | | | |
| | Collagenase | | | | | | | | | |
| | and | | | | | | | | | |
| | Hyaluronidase | | | | | | | | | |
| 2 | Enzymatic | 5.5 | 5.8 | 6 | 96.2 | 97.3 | 97.1 | 7 | 6 | 8 |
| | digestion | | | | | | | | | |
| | using | | | | | | | | | |
| | Collagenase | | | | | | | | | |
| | and | | | | | | | | | |
| | Hyaluronidase | | | | | | | | | |
| | along with | | | | | | | | | |
| | proteins | | | | | | | | | |
| | identified | | | | | | | | | |
| | from CB + MB | | | | | | | | | |
| | plasma lysate | | | | | | | | | |
| | (PRDX1 and | | | | | | | | | |
| | HSP70) | | | | | | | | | |

### Example 2: Production of Medium Sized MSCs

1. Expansion of hMSCs Up to Passage 3

The schematic process flow to produce the medium-sized MSCs is shown in FIG. 2. After isolation and adherence, the cultured cells were subjected to intermittent medium changes at every 48-72h intervals. This process was repeated up to 4-5 times depending on the growth of culture to achieve 70-80% confluency. Afterwards sub-culturing was carried out with the help of trypsin enzyme to enhance the growth or expansion of cells. For detachment of cells, PBS wash followed by trypsinization, and 2-3 min incubation was carried out by using 0.25% trypsin (GIBCO, Cat no. 25200056). Once the cells were found to be detached, immediately 0.5 ml fetal bovine serum was added to stop the enzymatic activity and cells were centrifuged at 1300 rpm for 5 min. The supernatant media and cell samples were taken for mycoplasma and sterility quality control (QC) testing. The cell viability, cell count, and cell characterization were performed to check the quality of cells. The cells were reseeded in T75 flask (cell density: about 5000-11,000/cm²) for further expansion and were monitored for its attachment status, cell morphology, cell growth and microbial contamination, if any. The remaining cells were cryopreserved for cells for future use. The similar procedure was followed up to passage 3 (P3) expansion and cryopreservation.

### 2. Cell Quality and Characterization

At the level of P3, cells were characterized and evaluated with their identification markers and cultured conditions to confirm the healthy state of cells (Table 4).

Cells were examined with the routine QC testing protocols to confirm the pure population.

TABLE 4
Characterization of cells and quality assessment of isolated and expanded
population at P3.

| S. No. | Test | Specification | Test Results |
|---|---|---|---|
| 1 | Cell number | Over 1000,000 | 1000,000 cells/vial |
| | | cells/vial | |
| 2 | Cell viability | ≥80% Dye- | 99% |
| | | Excluding cells | |
| 3 | Microbial Sterility | Negative/no growth | Negative/no growth |
| 4 | Endotoxin | <3 EU/ml | <0.750 EU/ml |
| 5 | Mycoplasma | Negative | Negative |
| 6 | Non -viable purities | <1 g/dL | 0.375 g/dL |
| 7 | Karyotyping | Metaphase | 46, XX or 46, |
| | | stage of | XY i.e Normal |
| | | mitosis | female/male |
| | | | karyotype pattern |
| 8 | Immuno- | >80% (For positive | 99.696% CD90 |
| | phenotyping | markers) | 99.223% CD73 |
| | | | 98.050% CD105 |
| | | <10% (For negative | 0.408% HLADR |
| | | markers) | 2.893% CD34 |
| | | | 3.317% CD45 |

### 3. Microfluidic Characterization

### Single Cell Droplet Encapsulation and Identification of Characteristic Population

At the stage of P3, cells (heterogeneous population of MSCs) were processed for Microfluidics analysis. For droplet encapsulation the single cell suspension (1×10⁶ cells/ml) was made and stained with desired markers. The staining procedure followed is briefed below:
1. 1×10⁶ cells/ml media was taken in a 1.5 ml Eppendorf tube.
2. The cells were centrifuged at 180 g for 5 min at 4° C.
3. The media was discarded without disturbing the pellet and the cells were resuspended in fresh 1 ml PBS.
4. The cells were mixed with gentle pipetting so that single cell suspension would be made.
5. 500 μl of suspension solution were centrifuged and washed with dPBS and centrifuged at 180 g for 5 min at 4° C. temperature.
6. The pellet was resuspended into 1 ml PBS.
7. The 1 ml solution was divided into 2 parts—400 μl and 600 μl.
8. 2 μl of CFSE stain (cell viability stain) and 5 μl CD73/90 stain (MSC marker stain) were added to 400 and 600 μl cell suspension in dPBS and incubated at 37° C. for 30 min.
9. After 30 min incubation, cells were washed, centrifuged, and resuspended from both the vials into 250 μl of PBS together.
10. 25 μl Opti-prep density gradient solution was added into the vial.
11. Cells were diluted by 1:10 ratio and flown into droplet generation device for encapsulation.
12. Cells were immobilized into array device and imaged for CFSE, CD73 and CD90 stains.

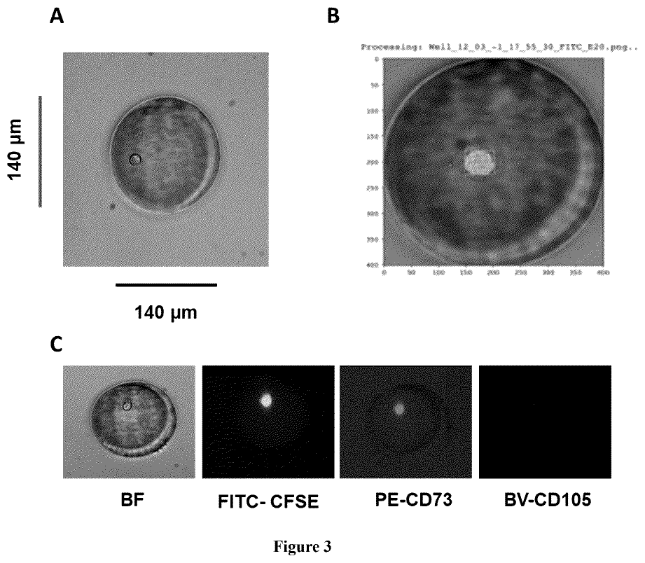

After staining the cells were washed, centrifuged (180 g, 5 min, 4° C.) and with gentle pipetting resuspended in media with 1/10^{th} Opti-prep before droplet encapsulation. 1 nanoliter sized droplets were generated which was sufficient to allow cells to replicate at least 3-4 times (FIG. 3A-B). These tiny droplets were spatially defined and lodged in nano-wells. In microfluidics platform (Droplet Microfluidic Platform), 1000 droplets are dispensed in a nano-well device. The cells were sorted out at 150 nanoliter/sec flow rate and imaging was done with automated fluorescence microscope. The nano-wells on chip were physically separated to prevent any cross talk and helpful to automated retrieval of selected cells. The microscope can automatically position the device to defined nano-well co-ordinates.

Bright-field and multiple fluorescence images were collected (FIG. 3C). The cell size (diameter), viability (CFSC) and surface markers (CD73/90) positivity were analyzed. The analysis was done by artificial intelligence (AI) based tool trained for size-based identification of cells. After screening and imaging, the device was taken out and kept for incubation in a CO₂ incubator until further processing. Image analysis was done with AI tool, which has been retrained to identify cells in images. The model marked out the cell boundaries and locations and provided the information of cell size and total fluorescence intensity. Programs used in AI were developed in python using open-source standard libraries.

Size distribution and Mean fluorescence Intensity measurement—After encapsulation by AI tool analysis, the varied size of the cells in droplets were measured. Microscopy analysis confirmed distinct differences in cell size among the encapsulated populations (Table 5). The average cell sizes were measured to be 12.2±1.9 μm, 19.6±2.6 μm, 35.8±6.7 μm for the small, medium-size, and large MSC subpopulations, respectively. The small and large subpopulation each contained 40 and 2% of the cells in total population, respectively. The majority was sorted into medium size subpopulation, comprising 58% of the total cells.

TABLE 5
Segregation of three population based on size (diameter) and its equivalent
mean fluorescence intensity (MFI) values of identification markers.

| | Size of cells | CD73 (MFI) | CD90 (MFI) |
|---|---|---|---|
| Population | (Avg ± SD) | (Avg ± SD) | (Avg ± SD) |
| Small | 12.2 ± 1.9 μm | 5.67 ± 0.9 | 5.9 ± 0.8 |
| Medium | 19.6 ± 2.6 μm | 6.0 ± 0.8 | 6.5 ± 0.7 |
| Large | 35.8 ± 6.7 μm | 6.0 ± 0.15 | 6.8 ± 0.8 |

With the present exercise, the three subsets of populations (based on its size) were recognized where the expression of identification markers was measured. It was hypothesized to obtain a correlation between mean fluorescence intensity values with their respective cell size, however no significant changes were recorded in this reference.

### Retrieval of Three Subset Populations

Once the cells were recorded, cells were retrieved with the AI tool for automated size-based sorting and collected in vials containing 1 ml PBS based on their referenced size groups i.e., small, medium, and large. Immediately after collection, cells were centrifuged at 180 g, 5 min, 4° C. and pellets were collected in cultured DMEM media.

The similar microfluidics sorting and measuring was repeated 5-6 times in a same day and cells were pooled. Approx 1000-3000 cells were there in each vial and seeded in 96 well plate for further expansion. Media was changed in every 48 h. Once the optimum number of cells was achieved, the cells were passaged to newer T25 flask followed to T75 and further expansion up was done using collagen coated microcarriers (Cytodex 3) and bioreactor to scale up the cells up to P6 (cell bank).

### Example 3: Characterization of Medium Sized MSCs

### Microarray Analysis

After the confirmation about the qualified target medium sized population, the transcriptome of qualified cells was obtained via mRNA sequencing using Illumina platform. For microarray analysis, 500 ng to 1000 ng total RNA was isolated from the 10⁶ cells using Trizol lysis and measured using Qubit fluorometer, in nuclease free water or low-TE (10 mM Tris, 0.1 mM EDTA). The parameter concentration ˜50 ng/μl with a minimum of 20 μl of sample was fixed to get good yield. The RNA integrity number (RIN)≥8, with 2100 was screened with Bioanalyzer Un-degraded (DNase treated).

Library Preparation: A modified NEBNext RNA Ultra II directional protocol was used to prepare the libraries for mRNA sequencing. In the first step the poly-A containing mRNA molecules were selectively purified using oligo-dT attached magnetic beads. Following purification, the mRNA was fragmented using divalent cations under elevated temperature. Next, the cDNA was synthesized using Reverse transcriptase and random hexamers in a first strand synthesis reaction. Subsequently, the cDNA was converted to double stranded cDNA where Uracil was added instead of Thymine. The strand specificity was preserved by a USER enzyme-based digestion of the second strand thereby leaving one functional strand, which maps to the DNA strand which it was transcribed from. The USER digested single strand molecules were enriched and indexed in a limited cycle PCR followed by AMPure bead purification to create final cDNA library for sequencing. Sequencing Prepared libraries were sequenced on Illumina HiSeqX/Novaseq to generate 2×150 bp reads/sample. Up to 75% of the sequenced bases were of Q30 value. Sequenced data were processed to generate FASTQ files and uploaded on the FTP server.

TABLE 6
Information and data extracting with Microarray data.

| | | | Up- | Upregulated | Upregulated |
|---|---|---|---|---|---|
| | | | regulated | (highly | (highly |
| | | | genes after | expressed) | expressed) |
| | Total | Statistical | significant | genes in | genes in |
| Total | up- | sig- | criteria | Small | Medium |
| Genes | regulated | nificance | (P ≤ | sized | sized |
| Identified | genes | criteria | 0.001) | population | population |
| 57773 | 965 | P ≤ 0.001 | 110 | 34 | 64 |

TABLE 7
List of highly expressed genes in medium sized population with details.

| S. | | Gene | | | | | base | log2Fold- | Small | Medium |
|---|---|---|---|---|---|---|---|---|---|---|
| NO | Gene ID | symbol | Chr | Start | End | Strand | Mean | Change | Sized | sized |
| 1 | ENSG00000111799 | COL12A1 | chr 6 | 75794042 | 75915767 | − | 26780.93422 | 17.81225464 | 0 | 53561.87 |
| 2 | ENSG00000115461 | IGFBP5 | chr 5 | 2.18E+08 | 2.18E+08 | − | 13548.38335 | 16.82919276 | 0 | 27096.77 |
| 3 | ENSG00000186340 | THBS2 | chr 6 | 1.7E+08 | 1.7E+08 | − | 12835.68502 | 16.75123419 | 0 | 25671.37 |
| 4 | ENSG00000166923 | GREM1 | chr 15 | 33010175 | 33026870 | + | 11399.2204 | 16.5800131 | 0 | 22798.44 |
| 5 | ENSG00000170558 | CDH2 | chr 18 | 25530930 | 25757410 | − | 6438.397316 | 15.75587063 | 0 | 12876.79 |
| 6 | ENSG00000013297 | CLDN11 | chr 3 | 1.7E+08 | 1.71E+08 | + | 6056.947573 | 15.66776196 | 0 | 12113.9 |
| 7 | ENSG00000133110 | POSTN | chr 13 | 38136720 | 38172981 | − | 6017.419103 | 15.6583161 | 0 | 12034.84 |
| 8 | ENSG00000060718 | COL11A1 | chr 1 | 1.03E+08 | 1.04E+08 | − | 5557.307703 | 15.54356 | 0 | 11114.62 |
| 9 | ENSG00000140937 | CDH11 | chr 16 | 64977656 | 65160015 | − | 5120.122817 | 15.42535498 | 0 | 10240.25 |
| 10 | ENSG00000108691 | CCL2 | chr 17 | 32582304 | 32584222 | + | 4925.247456 | 15.36937405 | 0 | 9850.495 |
| 11 | ENSG00000003989 | SLC7A2 | chr 8 | 17354597 | 17428082 | + | 4537.473158 | 15.25106961 | 0 | 9074.946 |
| 12 | ENSG00000144810 | COL8A1 | chr 3 | 99357319 | 99518070 | + | 4034.67101 | 15.08163537 | 0 | 8069.342 |
| 13 | ENSG00000152661 | GJA1 | chr 6 | 1.22E+08 | 1.22E+08 | + | 4011.744497 | 15.07341424 | 0 | 8023.489 |
| 14 | ENSG00000091986 | CCDC80 | chr 3 | 1.12E+08 | 1.12E+08 | − | 3824.77483 | 15.00456088 | 0 | 7649.55 |
| 15 | ENSG00000163017 | ACTG2 | chr 2 | 74119441 | 74146992 | + | 3299.836738 | 14.79158546 | 0 | 6599.673 |
| 16 | ENSG00000169604 | ANTXR1 | chr 2 | 69240310 | 69476459 | + | 3242.125171 | 14.76613117 | 0 | 6484.25 |
| 17 | ENSG00000187720 | THSD4 | chr 15 | 71389291 | 72075722 | + | 3212.874103 | 14.75305612 | 0 | 6425.748 |
| 18 | ENSG00000115963 | RND3 | chr 2 | 1.51E+08 | 1.51E+08 | − | 2208.455661 | 14.2122414 | 0 | 4416.911 |
| 19 | ENSG00000101825 | MXRA5 | chr X | 3226606 | 3264682 | − | 2200.945251 | 14.2073269 | 0 | 4401.891 |
| 20 | ENSG00000182667 | NTM | chr 11 | 1.31E+08 | 1.32E+08 | + | 2177.228169 | 14.19169659 | 0 | 4354.456 |
| 21 | ENSG00000187079 | TEAD1 | chr 11 | 12695969 | 12966298 | + | 2069.710729 | 14.11863471 | 0 | 4139.421 |
| 22 | ENSG00000137693 | YAP1 | chr 11 | 1.02E+08 | 1.02E+08 | + | 2023.067133 | 14.08575047 | 0 | 4046.134 |
| 23 | ENSG00000173546 | CSPG4 | chr 15 | 75966663 | 76005189 | − | 2007.651029 | 14.07471503 | 0 | 4015.302 |
| 24 | ENSG00000255248 | RP11- | chr 11 | 1.22E+08 | 1.22E+08 | − | 1959.82158 | 14.03992958 | 0 | 3919.643 |
| | | 166D19.1 | | | | | | | | |
| 25 | ENSG00000146242 | TPBG | chr 6 | 83072923 | 83080545 | + | 1956.659302 | 14.03759989 | 0 | 3913.319 |
| 26 | ENSG00000078098 | FAP | chr 2 | 1.63E+08 | 1.63E+08 | − | 1917.921401 | 14.00875153 | 0 | 3835.843 |
| 27 | ENSG00000058085 | LAMC2 | chr 1 | 1.83E+08 | 1.83E+08 | + | 1742.414991 | 13.87029918 | 0 | 3484.83 |
| 28 | ENSG00000139329 | LUM | chr 12 | 91496406 | 91505608 | − | 1580.743545 | 13.72981723 | 0 | 3161.487 |
| 29 | ENSG00000074590 | NUAK1 | chr 12 | 1.06E+08 | 1.07E+08 | − | 1479.155376 | 13.63398954 | 0 | 2958.311 |
| 30 | ENSG00000169213 | RAB3B | chr 1 | 52373628 | 52456436 | − | 1459.39114 | 13.61458294 | 0 | 2918.782 |
| 31 | ENSG00000164294 | GPPX8 | chr 5 | 54455946 | 54462899 | + | 1453.066585 | 13.60831729 | 0 | 2906.133 |
| 32 | ENSG00000134762 | DSC3 | chr 18 | 28569974 | 28622781 | − | 1428.954218 | 13.58417668 | 0 | 2857.908 |
| 33 | ENSG00000128606 | LRRC17 | chr 7 | 1.03E+08 | 1.03E+08 | + | 1404.446566 | 13.55921925 | 0 | 2808.893 |
| 34 | ENSG00000183098 | GPC6 | chr 13 | 93879095 | 95059655 | + | 1393.378594 | 13.54780506 | 0 | 2786.757 |
| 35 | ENSG00000077942 | FBLN1 | chr 22 | 45898118 | 45997015 | + | 1362.543687 | 13.51552378 | 0 | 2725.093 |
| 36 | ENSG00000154734 | ADAMTS1 | chr 21 | 28208066 | 28217728 | − | 1336.457596 | 13.48763308 | 0 | 2672.915 |
| 37 | ENSG00000185551 | NR2F2 | chr 15 | 96869167 | 96883492 | + | 1331.71418 | 13.4825036 | 0 | 2663.428 |
| 38 | ENSG00000112902 | SEMA5A | chr 5 | 9035138 | 9546187 | − | 1318.2745 | 13.46787024 | 0 | 2636.549 |
| 39 | ENSG00000145147 | SLIT2 | chr 4 | 20254883 | 20622184 | + | 1317.088645 | 13.46657191 | 0 | 2634.177 |
| 40 | ENSG00000135111 | TBX3 | chr 12 | 1.15E+08 | 1.15E+08 | − | 1299.300834 | 13.44695538 | 0 | 2598.602 |
| 41 | ENSG00000166173 | LARP6 | chr 15 | 71123863 | 71146498 | − | 1290.999855 | 13.43770891 | 0 | 2582 |
| 42 | ENSG00000170961 | HAS2 | chr 8 | 1.23E+08 | 1.23E+08 | − | 1244.356259 | 13.37462077 | 0 | 2488.713 |
| 43 | ENSG00000178882 | FAM101A | chr 12 | 1.24E+08 | 1.25E+08 | + | 1240.008127 | 13.37957086 | 0 | 2480.016 |
| 44 | ENSG00000185070 | FLRT2 | chr 14 | 85996488 | 86095034 | + | 1181.901275 | 13.31033226 | 0 | 2363.803 |
| 45 | ENSG00000103855 | CD276 | chr 15 | 73976307 | 74006859 | + | 1168.461595 | 13.29683339 | 0 | 2336.923 |
| 46 | ENSG00000176046 | NUPR1 | chr 16 | 28548606 | 28550495 | − | 1165.694602 | 13.29041301 | 0 | 2331.389 |
| 47 | ENSG00000104067 | TJP1 | chr 15 | 29991571 | 30261068 | − | 1131.304833 | 13.24721176 | 0 | 2262.61 |
| 48 | ENSG00000163629 | PTPN13 | chr 4 | 87515468 | 87736324 | + | 1111.540598 | 13.22178521 | 0 | 2223.081 |
| 49 | ENSG00000072840 | EVC | chr 4 | 5712924 | 5830772 | + | 1087.42823 | 13.19014541 | 0 | 2174.856 |
| 50 | ENSG00000026559 | KCNG1 | chr 20 | 49620193 | 49639666 | − | 1038.808211 | 13.12415591 | 0 | 2077.616 |
| 51 | ENSG00000149090 | PAMR1 | chr 11 | 35453370 | 35551848 | − | 974.376804 | 13.03178018 | 0 | 1948.754 |
| 52 | ENSG00000154553 | PDLIM3 | chr 4 | 1.86E+08 | 1.86E+08 | − | 947.8927286 | 12.99202501 | 0 | 1895.785 |
| 53 | ENSG00000159167 | STC1 | chr 8 | 23699428 | 23712320 | − | 942.3587427 | 12.98357776 | 0 | 1884.717 |
| 54 | ENSG00000168077 | SCARA3 | chr 8 | 27491385 | 27534293 | + | 930.1049168 | 12.96469523 | 0 | 1860.21 |
| 55 | ENSG00000116132 | PRRX1 | chr 1 | 1.71E+08 | 1.71E+08 | + | 872.3933495 | 12.87228243 | 0 | 1744.787 |
| 56 | ENSG00000063660 | GPC1 | chr 2 | 2.41E+08 | 2.41E+08 | + | 862.5112318 | 12.85584723 | 0 | 1725.022 |
| 57 | ENSG00000139874 | SSTR1 | chr 14 | 38677204 | 38682272 | + | 855.3961071 | 12.84389687 | 0 | 1710.792 |
| 58 | ENSG00000067048 | DDX3Y | chr Y | 15016019 | 15032390 | + | 847.0951282 | 12.82982851 | 0 | 1694.19 |
| 59 | ENSG00000079931 | MOXD1 | chr 6 | 1.33E+08 | 1.33E+08 | − | 789.3935609 | 12.72803305 | 0 | 1578.767 |
| 60 | ENSG00000117152 | RGS4 | chr 1 | 1.63E+08 | 1.63E+08 | + | 773.1768879 | 12.69810569 | 0 | 1546.354 |
| 61 | ENSG00000121068 | TBX2 | chr 17 | 59477257 | 59486827 | + | 770.409895 | 12.69293352 | 0 | 1540.82 |
| 62 | ENSG00000128510 | CPA4 | chr 7 | 1.3E+08 | 1.3E+08 | + | 768.8287561 | 12.68996964 | 0 | 1537.658 |
| 63 | ENSG00000167779 | IGFBP6 | chr 12 | 53491220 | 53496129 | + | 767.642902 | 12.68774273 | 0 | 1535.286 |
| 64 | ENSG00000171724 | VAT1L | chr 16 | 77822427 | 78014004 | + | 754.203222 | 12.66226122 | 0 | 1508.406 |

TABLE 8
Importance of unique identified markers (Top 5) in medium sized
population in reference to Autoimmune disease treatment:

| | | | | | Clinical |
|---|---|---|---|---|---|
| | | | | | correlation- |
| | | | | | with |
| | | | Clinical | Clinical | inflammatory |
| | | Clinical | correlation- | correlation- | bowel disease |
| | | correlation- | with Type I | with multiple | & Sjogren's |
| Gene | Biological | with Arthritis | Diabetes & | sclerosis & | syndrome & |
| name | Function | & specific role | specific role | specific role | specific role |
| COL | Skeletal | Clinical | — | Clinical; in | — |
| 12 A1 | system | (PMID: 27701424) | | thyroid- | |
| | development, | Specific | | associated | |
| | extracellular | role | | ophthalmopathy | |
| | matrix | Enlisted in | | (PMID: 34078122) | |
| | structural | Chondro- | | | |
| | constituent, | specific | | | |
| | collagen | genes; | | | |
| | fibril | indicate | | | |
| | organization, | MSCs | | | |
| | chondrogenic | inherent | | | |
| | capacity | higher | | | |
| | | chondrogenic | | | |
| | | capacity | | | |
| | | with | | | |
| | | efficient | | | |
| | | matrix | | | |
| | | organization. | | | |
| IGFBP5 | Matrix | IGF-1 | No report | No report | Clinical; |
| | protein, | Clinical | Specific | Specific | (PMID: 24379630) |
| | Regulation | correlate | role: | role: | |
| | of | (PMID: 21617253). | Could be | IGFBP5 as | |
| | smooth | With | used as a | an | |
| | muscle | IGFBP5 - | marker for | inhibitory | |
| | cell | No report | gestational | binding | |
| | migration; | Specific | diabetes | protein for | |
| | and | role: As | mellitus | IGF-1 has | |
| | regulation | IGF-I | | role in | |
| | of smooth | modulator, | | amyotrophic | |
| | muscle | it controls | | lateral | |
| | cell | cartilage | | sclerosis. | |
| | proliferation | formation | | Also plays | |
| | | and | | crucial role | |
| | | inhibiting | | in drive | |
| | | degeneration | | fibrosis | |
| | | i.e., | | and tissue | |
| | | impact on | | remodeling | |
| | | cartilage | | with | |
| | | metabolism | | Pulmonary | |
| | | | | fibrosis. | |
| THBS2 | Glycoprotein | Clinical | Clinical | — | — |
| | expressed | (PMID: 23843355; | PMID: 36335340; | | |
| | in the | PMID: 30592390) | PMID: 34183428 | | |
| | extracellular | Specific role: | Specific role: | | |
| | matrix | Marker to | TSP2-deficient | | |
| | (ECM), | predict | mice developed | | |
| | role tissue | differentiation | obesity and | | |
| | remodeling, | potential of | hyperglycemia. | | |
| | potent | MSCs for | Mechanistically | | |
| | inhibitor | cartilage | high glucose | | |
| | of tumor | regeneration | increased | | |
| | growth | therapy; | activation of | | |
| | and | Endogenous | the hexosamine | | |
| | angiogenesis | regulator of | pathway and | | |
| | | angiogenesis | nuclear factor- | | |
| | | and | κB signaling to | | |
| | | autoimmune | elevate TSP2 | | |
| | | inflammation in | expression. | | |
| | | the synovium | | | |
| GREM1 | Member | No report | — | — | — |
| | of the | Specific role: | | | |
| | BMP | Gremlin 1 | | | |
| | (bone | outlines a | | | |
| | morphogenic | resident of | | | |
| | protein) | osteochondroreticular | | | |
| | antagonist | stem cells | | | |
| | family, | in the bone | | | |
| | role in | marrow | | | |
| | regulating | | | | |
| | organogenesis, | | | | |
| | tissue | | | | |
| | differentiation | | | | |
| | inhibitor | | | | |
| | in the | | | | |
| | TGF-β | | | | |
| | signaling | | | | |
| | pathway | | | | |
| CDH2 | Also | No report | Clinical; | — | — |
| | refer as | Specific role | (PMID: 35363774) | | |
| | NCAD, | in cartilage | | | |
| | member | regeneration & | | | |
| | of the | chondrogenesis | | | |
| | cadherin | predictability: | | | |
| | superfamily, | Mediates cell- | | | |
| | plays | cell interactions, | | | |
| | role in | regulates β- | | | |
| | formation | catenin (βcat) | | | |
| | of | signaling, and | | | |
| | cartilage | promotes the | | | |
| | and bone | chondrogenic | | | |
| | | differentiation | | | |
| | | of mesenchymal | | | |
| | | lineage cells | | | |

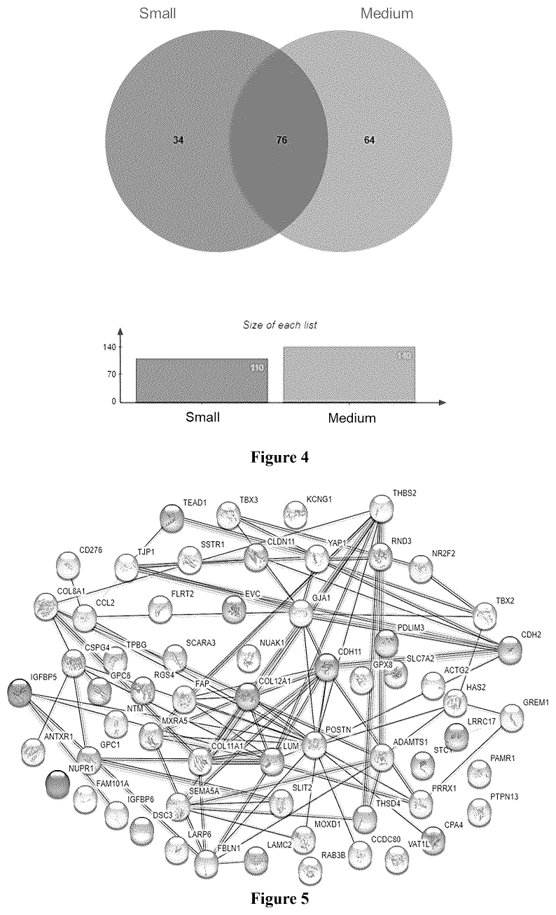

Sixty-four genes were found to be highly expressed in the medium-sized MSCs as compared to small sized MSCs with 34 genes (Table 6 and Table 7; FIG. 4). Among the 64 genes, the top five genes COL12 A1, IGFBP5, THBS2, GREM1 CDH2, were unique markers of medium sized population and also predicted to be clinically associated with chondrogenesis and cartilage regeneration (FIG. 5, Table 8) using the STRING online tool.

### Cryopreservation and Stability Studies of the Medium-Sized MSCs

Around one billion expanded medium sized MSCs at the P6 stage were cryopreserved in 3 sets of cryovials using medium containing 90% of FBS and 10% DMSO (34 vials, 10 million MSCs each), 90% of HSA and 10% DMSO (33 vials, 10 million MSCs each), and 90% cord blood and maternal blood plasma along with 10% DMSO (33 vials, 10 million, MSCs each) at a temperature below −150° C. in liquid nitrogen tank, under vapor phase conditions. After the completion of 6 month, 1 years, and 2 years, 1 vial from each sample was thawed at 37° C. in water bath and was further analyzed for Cell count, Cell viability, doubling time, growth kinetics, Annexin V, Cell Cycle analysis was performed to check the status of the cell stock.

TABLE 9
Stability/Reprocess data (CB + MB plasma with DMSO)

| | Stability/ | Stability/ | Stability/ |
|---|---|---|---|
| | Reprocess | Reprocess | Reprocess |
| | data at 6 months. | data at 1 year. | data at 2 years. |
| | (Average | (Average | (Average |
| Test | value of 3 | value of | value of 3 |
| Parameter | samples) | 3 samples) | samples) |
| Cell Count | 8.1 × 10\hat{ }6 ± 2.9 | 7.9 × 10\hat{ }6 ± 0.9 | 7.2 × 10\hat{ }6 ± 1.2 |
| Cell Viability | 95% | 93% | 91% |
| Annexin V | 90.2 ± 2.3% | 92.62 ± 1.3% | 92.0 ± 1.8% |
| Cell Cycle | G0/G1 phase | G0/G1 phase | G0/G1 phase |
| CD73/90/105 | 97.25 ± 1.1% | 95.72 ± 2.7% | 92.67 ± 3.0% |
| Cd3/19/56 | 0.32 ± 3.2% | 0.43 ± 2.2% | 0.60 ± 1.5% |
| CD34/45, | 0.6% HLADR | 0.7% HLADR | 0.5% HLADR |
| HLADR | | | |

### Advantages of the Present Disclosure

The present disclosure provides a method for producing MSCs with therapeutic potential with the following advantages.
a) The specific size (19.6±2.6 μm), marker positivity (CD73/90/105) and gene expression profile of the MSCs make them unique with significant therapeutic potential.
b) The MSCs can be used as a cellular therapeutic tool for autoimmune disease (like RA) treatment due to their potent suppressive action to inhibit proinflammatory cells from both the innate and adaptive immune system.
c) The MSCs can be provided as off the shelf, allogenic therapy product.
d) The MSCs obtained using the disclosed method exhibits improved stability and viability even after cryopreserving up to 2 years.
e) The medium sized cells selected have shown higher expression of genes, which are responsible for the regeneration of cartilage, bone, etc for the tissue regeneration process.
f) The microfluidics based MSCs cell sorting, and expansion thereafter gives much more predictability for the end use of the cells in therapeutic applications.

I/We claim:

1. A method of producing mesenchymal stem cells (MSCs) for therapeutic application, the method comprising:
(a) culturing a heterogeneous population of MSCs in a culture medium for a period in a range of 20 to 35 days;
(b) staining the MSCs to obtain a heterogeneous population of stained MSCs;
(c) adding droplet encapsulation media, comprising a density gradient solution to the heterogeneous population of stained MSCs, to obtain a population of microfluidic droplets, wherein each droplet preferably comprises a single MSC;
(d) providing the population of microfluidic droplets obtained from step (c) to a microfluidics device; and
(e) identifying and selecting a population of homogeneous MSCs having medium size in the range of 15 to 30 μm;
wherein the population of homogeneous MSCs obtained from step (e) are viable MSCs, expressing MSC specific markers selected from CD 73, CD 90, and combinations thereof; and exhibiting increased expression of genes selected from a group consisting of COL12A gene, IGFBP5 gene, THBS2 gene, GREM1 gene, CDH2 gene, and
combinations thereof.

2. The method as claimed in claim 1, wherein the step (e) further comprises:
imaging the MSCs in the population of microfluidic droplets to record the size of the MSCs; and subjecting the population of microfluidic droplets to automated size-based sorting to obtain a population of homogeneous MSCs having medium size.

3. The method as claimed in claim 1, wherein the step (a) further comprises:
reseeding the heterogeneous population of MSCs at a cell density in the range of 5000 to 11,000/cm² and expanding the heterogeneous population of MSCs up to passage 3.

4. The method as claimed in claim 1, wherein the heterogeneous population of MSCs in step (b) are stained using a cell viability stain or MSC marker stain; and wherein the heterogeneous population of MSCs is having a concentration in the range of 0.5 to 5×10⁶ cells/ml, preferably having a concentration of 1×10⁶ cells/ml.

5. The method as claimed in claim 1, wherein the size of the microfluidic droplet is in the range 100 to 200 micron; and each of the microfluidic droplet is sufficient to allow the MSCs to replicate 3 to 4 times.

6. The method as claimed in claim 1, wherein the population of microfluidic droplets are produced using a droplet generation device.

7. The method as claimed in claim 1, wherein the step (d) further comprising, analyses of the microfluidic droplets at a flow rate in the range of 100 to 200 nanolitre/sec, preferably at a flow rate of 150 nanolitre/sec.

8. The method as claimed in claim 1, wherein the method further comprises, expanding the population of homogeneous MSCs having medium size up to passage 6 or up to passage 20 in culture; and optionally cryopreserving the MSCs.

9. The method as claimed in claim 4, wherein the cell viability stain is selected from carboxyfluorescein diacetate succinimidyl ester (CFSE), trypan blue, propidium iodide (PI), or combinations thereof; and the MSC marker stain is selected from CD73, CD90, CD105, or combinations thereof.

10. The method as claimed in claim 1, wherein the culture medium is selected from Dulbecco's Modified Eagle Medium (DMEM), Iscove's Modified Dulbecco's Medium (IMDM), DMEM-F12, F12, Minimum Essential Medium a (ALPHA-MEM), or combinations thereof.

11. The method as claimed in claim 1, wherein the heterogeneous population of MSCs is derived from human umbilical cord tissue of single donor or multiple donors.

12. The method as claimed in claim 11, wherein the heterogeneous population of MSCs is obtained by treating the human umbilical cord tissue with at least one enzyme selected from collagenase, hyaluronidase, proteins identified from CB+MB plasma lysate (Peroxiredoxin 1-PRDX1 and Heat Shock Protein-70-HSP70), or mixtures thereof.

13. The method as claimed in claim 2, wherein the automated size-based sorting is performed using an artificial intelligence-based tool trained for size-based identification of cells.

14. The method as claimed in claim 1, wherein the homogeneous population of MSCs comprises 58% to 60% of MSCs having medium size.

15. A composition comprising the MSCs obtained by the method as claimed in claim 1; and an excipient; wherein the excipient is selected from DMEM, human serum albumin (HSA), dimethyl sulfoxide (DMSO), fetal bovine serum (FBS), cord blood and maternal blood plasma or combinations thereof.

16. A formulation comprising the MSCs obtained by the method as claimed in claim 1; and a pharmaceutically acceptable carrier.

17. The formulation as claimed in claim 16, wherein the pharmaceutically acceptable carrier is selected from ringer lactate solution, saline, dextrose, heparin, or combinations thereof.

18. A method of treating a disease in a subject, comprising administering the MSCs obtained from the method as claimed in claim 1 to a subject.

19. The method as claimed in claim 18, wherein the MSCs is allogenic to the subject.

20. The method as claimed in claim 18, wherein the disease is selected from a group consisting of autoimmune diseases (Rheumatoid Arthritis (RA), Acquired Aplastic Anemia, Acquired Hemophilia, Agammaglobulinemia, Alopecia Areata, Ankylosing Spondylitis (AS), Anti-NMDA Receptor Encephalitis, Antiphospholipid Syndrome (APS), Arteriosclerosis, Autoimmune Addison's Disease (AAD), Autoimmune Autonomic Ganglionopathy (AAG), Autoimmune Encephalitis (AE)/Acute Disseminated Encephalomyelitis (ADEM), Autoimmune Gastritis, Autoimmune Hemolytic Anemia (AIHA), Autoimmune Hepatitis, Autoimmune Hyperlipidemia, Autoimmune Hypophysitis/Lymphocytic Hypophysitis, Autoimmune Inner Ear Disease (AIED), Autoimmune Lymphoproliferative Syndrome (ALPS), Autoimmune Myelofibrosis (AIMF), Autoimmune Myocarditis, Autoimmune Oophoritis, Autoimmune Pancreatitis (AIP), Autoimmune Polyglandular Syndromes (APS), Autoimmune Progesterone Dermatitis (APD), Autoimmune Retinopathy (AIR), Autoimmune Sudden Sensorineural Hearing Loss, Balo Disease/Concentric Sclerosis, Behçet's Disease, Birdshot Chorioretinopathy/Birdshot Uveitis, Bullous Pemphigoid, Castleman Disease, Celiac Disease, Chagas Disease, Chronic Inflammatory Demyelinating Polyneuropathy (CIDP), Chronic Autoimmune Urticaria, Churg-Strauss Syndrome/Eosinophilic Granulomatosis with Polyangiitis (EGPA), Cogan's Syndrome (CS), Cold Agglutinin Disease (CAD), Crest Syndrome, Crohn's Disease, Stricturing Crohn's Disease, Cronkhite-Canada Syndrome (CCS), Cryptogenic Organizing Pneumonia (COP), Dermatitis Herpetiformis (DH), Dermatomyositis, Diabetes, Type 1 (TID), Discoid Lupus Erythematosus (DLE), Dressler's Syndrome/Post myocardial Infarction/Post pericardiotomy Syndrome, Eczema/Atopic Dermatitis, Eosinophilic Fasciitis, Erythema Nodosum, Essential Mixed Cryoglobulinemia, Evans Syndrome, Fibrosing Alveolitis/Idiopathic Pulmonary Fibrosis (IPF), Giant Cell Arteritis/Temporal Arteritis/Horton's Disease, Giant Cell Myocarditis, Glomerulonephritis (GN), Goodpasture's Syndrome/Anti-Gbm/Anti-Tbm Disease, Granulomatosis With Polyangiitis (GPA)/Wegener's Granulomatosis, Graves' Disease (GD), Guillain-Barrè Syndrome (GBS), Hashimoto's Thyroiditis/Autoimmune Thyroiditis, Henoch-Schölein Purpura (HSP)/Iga Vasculitis, Hidradenitis Suppurativa, Hurst's Disease/Acute Hemorrhagic Leukoencephalitis (AHLE), Hypogammaglobulinemia, Iga Nephropathy/Berger's Disease, Immune-Mediated Necrotizing Myopathy (IMNM), Immune Thrombocytopenia (Itp)/Autoimmune Thrombocytopenia Purpura, Inclusion Body Myositis (IBM), Igg4-Related Sclerosing Disease (ISD), Interstitial Cystitis, Juvenile Idiopathic Arthritis (Jia)/Adult-Onset Still's Disease, Juvenile polymyositis/Juvenile dermatomyositis/juvenile myositis, Kawasaki disease, Lambert-Eaton Myasthenic Syndrome (LEMS), Leukocytoclastic vasculitis, Lichen Planus, Lichen Sclerosus, Ligneous conjunctivitis, Linear Iga Disease (LAD), Lupus Nephritis (LN), Lyme Disease/Chronic Lyme Disease/Post-Treatment Lyme Disease Syndrome (PTLDS), Lymphocytic colitis/microscopic colitis, Lymphocytic hypophystitis/autoimmune hypophystitis, Ménière's Disease, Microscopic Polyangiitis (MPA)/ANCA-Associated Vasculitis, Mixed Connective Tissue Disease (MCTD), Mooren's ulcer, Mucha-Habermann disease, Multifocal motor neuropathy, Multiple Sclerosis (MS), Myalgic Encephalomyelitis/Chronic Fatigue Syndrome (ME/CFS), Myasthenia Gravis (MG), Narcolepsy, Neuromyelitis Optica/Devic's Disease, Ocular Cicatricial Pemphigoid, Opsoclonus-myoclonus syndrome (OMS), Palindromic Rheumatism, Paraneoplastic Cerebellar Degeneration (PCD), Paraneoplastic Pemphigus, Parry-Romberg Syndromeherth (PRS)/Hemifacial Atrophy (HFA)/Progressive Facial Hemiatrophy, Paroxysmal Nocturnal Hemoglobinuria (PNH), Peripheral uveitis/pars planitis, PANS/PANDAS, Parsonage-Turner Syndrome (PTS), Pemphigoid Gestationis (PG), Pemphigus Foliaceus, Pemphigus Vulgaris, Pernicious anemia, POEMS Syndrome, Polyarteritis Nodosa (PAN), Polymyalgia Rheumatica, Polymyositis, Postural Orthostatic Tachycardia Syndrome (Pots), Primary Biliary Cirrhosis (PBC), Primary Sclerosing Cholangitis (PSC), Psoriasis, Palmoplantar Pustulosis (PPP), Psoriatic Arthritis, Pulmonary fibrosis, idiopathic (IPF), Pure Red Cell Aplasia (PRCA), Pyoderma gangrenosum, Rasmussen's encephalitis, Raynaud's Syndrome, Reactive Arthritis, Reflex sympathetic dystrophy syndrome (RSD)/Complex regional pain syndrome (CRPS), Relapsing Polychondritis (RP), Restless leg syndrome (RLS)/Willis-Ekbom disease, Rheumatic Fever, Sarcoidosis, Schmidt Syndrome/Autoimmune Polyendocrine Syndrome Type II, Scleritis, Scleroderma, Sclerosing Mesenteritis/Mesenteric Panniculitis, Serpiginous choroidopathy, Sjögren's Syndrome, Stiff person syndrome (SPS), Small Fiber Sensory Neuropathy (SFSN), Small Fiber Sensory Neuropathy (SFSN), Systemic Lupus Erythematosus (SLE), Subacute bacterial endocarditis (SBE), Subacute cutaneous lupus, Susac's syndrome, Sydenham's Chorea, Sympathetic ophthalmia, Takayasu's arteritis (vasculitis), Testicular Autoimmunity, Tolosa-Hunt syndrome, Transverse myelitis™, Tubulointerstitial nephritis uveitis syndrome (TINU), Ulcerative Colitis, Undifferentiated Connective Tissue Disease, Uveitis, Vasculitis, VEXAS Syndrome, Vogt-Koyanagi-Harada syndrome (VKH), Osteoarthritis, AVN, vertebral compression factor, urethral stricture, Sjogren's syndrome and ureteric stricture), arthritis, Type I Diabetes, multiple sclerosis, inflammatory bowel diseases and acromegaly.
